(19) United States
(12) Patent Application Publication
(10) Pub. No.: US 2006/0115538 A1
(43) Pub. Date: Jun. 1, 2006

(54) Calcium lactate and whey permeate for lowering the triglyceride level

(76) Inventors: Jobst Krauskopf, UELZEN (DE); Christian Sowada, Nordestedt (DE)

Correspondence Address: SCULLY, SCOTT, MURPHY & PRESSER, 400 GARDEN CITY PLAZA, SUITE 300, GARDEN CITY, NY, 11530, US

(21) Appl. No.: 10/523,206

(22) PCT Filed: Jul. 22, 2003
(86) PCT No.: PCT/EP03/08004

§ 371 Date: Sep. 22, 2005

(30) Foreign Application Priority Data
Jul. 22, 2002 (DE) 102 33 229.0

(51) Int. Cl. A61K 9/50; A61K 35/20; A61K 31/19

(52) U.S. Cl. 424/490; 424/535; 514/557

(57) ABSTRACT

Disclosed is the combined use of calcium lactate and whey permeate for the prophylaxis or treatment of hypertriglyceridemia and/or symptoms thereof.

### AREA OF THE INVENTION

The present invention relates to a pharmaceutical composition comprising Ca-lactate and whey permeate for the treatment and/or prophylaxis of hypertriglyceridemia.

### BACKGROUND OF THE INVENTION

Coronary heart disease is the main cause of death in the industrialized nations. Thus, it is responsible for more than 500,000 deaths each year in the United States. It is estimated that coronary heart diseases directly and indirectly cost the United states more than 100 billion dollars each year. The primary cause of coronary heart diseases is arteriosclerosis, colloquially also referred to as hardening of the arteries, a disease characterized by the deposit of lipids (cholesterols and triglycerides) on the wall of arterial blood vessels, which leads to constriction of the arterial aperture and finally to hardening of the arteries

Arteriosclerosis, as it manifests itself in its principal clinical complication, coronary heart disease or ischemic heart disease, continues to be a main cause of death in industrialized nations. It is generally recognized that arteriosclerosis can begin with a local injury to the arterial endothelium, followed by penetration of circulating monocytes into the interior of the arterial wall, where they are charged with lipids originating from lipoprotein. At about the same time, there appears to occur a migration of arterial, smooth muscle cells from the intermediate layer to the inner layer and their growth in this location, together with the deposit of lipid and the accumulation of foam cells in the lesion. As the arteriosclerotic plaque develops, it increasingly occludes the affected blood vessel and can occasionally lead to ischemia, thrombosis, or infarction. It is therefore desirable that methods be provided for preventing the progression of arteriosclerosis in patients in need of such prevention.

Serum lipoproteins are the carriers for lipids in the bloodstream. They are classified according to their density: chylomicrons, very low density lipoproteins (VLDL), low density lipoproteins (LDL), intermediate density lipoproteins (IDL), and high density lipoproteins (HDL). About 50% to 70% of the cholesterol circulating in the blood is carried in the form of LDL. In contrast, about 25% of total cholesterol are found in HDL, whereas VLDL carries the bulk of plasma triglycerides and only about 10% to 15% of total cholesterol.

Chylomicrons are assembled in the intestinal wall from products of lipid digestion and are thereupon transported via the thoracicolymphatic system into the peripheral circulatory system. In the bloodstream, they are broken down by lipoprotein lipase (LPL) to free fatty acids and triglycerides (TG), which are utilized primarily for generation of energy by muscle and for storage in adipose tissue. The other serum lipoproteins are involved in the transport of endogenously synthesized lipid. Endogenous lipid transport begins when the liver secretes triglycerides and cholesterol into the plasma as VLDL. In the capillaries, the triglycerides of the VLDL are split by LPL into IDL and finally to LDL. Some of these particles are rapidly removed by the liver, by receptor-mediated endocytosis. The rest circulates for the greater part as LDL.

When cells die and cell membranes undergo turnover, cholesterol is continuously released into the plasma and bound to HDL. HDL promotes the removal of cholesterol from peripheral cells and promotes its transport back to the liver.

One speaks of hypertriglyceridemia (hyperlipemia) when the content of triglycerides in the blood serum is increased. This condition can play a role in artherogenesis and the development of coronary heart disease (Vega and Grundy, Adv. Exp. Med. 243, 311 (1989)). In addition, severe hypertriglyceridemia (>1,000 mg/dl) is connected with chylomicronemia and causes acute pancreatitis (see K. Soergel, Acute Pancreatitis, in Gastrointestinal Disease91, 3^{rd} edition (Sleisenger, M. H. and Fordtran, J. S., eds.), W. B. Saunders Company, Philadelphia, Pa., 1983, p. 1462-1485; and Brown, M. S. and Goldstein, J. L., Drugs used in the Treatment of Hyperlipoproteinemias, in Goodman and Gillman's, the Pharmacological Basis of Therapeutics 34, 7^{th} edition, (Macmillan Publishing Co., New York, 1985, S. 827-845). Serious increases in chylomicrons directly cause pancreatitis and these can be prevented by a decrease in triglycerides (U.S. Department of Health and Human Services, NIH-Publication No. 89-2925, p. 74-77, January 1989, “Report of the Expert Panel on Detection, Evaluation and Treatment of High Blood Cholesterol in Adults”). Moreover, it has become clear that the stroke risk in patients with coronary heart disease can be decreased by lowering the content of triglycerides in the plasma (D. Tanne, et al.; Circulation 2001, 104, 2892-2897). Furthermore, the life expectancy of type-2-diabetics is shortened by one third compared to non-diabetics. This presumably bears a direct relation to an increased triglyceride level (Diabety Care; 2001-24, 1335-1341). It is therefore desirable to provide a method for reducing plasma triglycerides in patients with hypertriglyceridemia.

Whey is obtained during the production of cheese from milk, such as, e.g., cow's milk, goat's milk, sheep's milk, buffalo's milk and camel's milk, after the casein present in milk has been precipitated. Depending of the manner of production, one distinguishes between sweet whey, which is formed as milk serum after enzymatic precipitation of casein with rennet, and sour whey, which is obtained after separation of the casein by acid precipitation. The pH-value boundary between sweet and sour whey is not defined entirely sharply and lies, in general, above or below a range of 5.6 to 5.9.

Whey contains all water-soluble components of milk, inasmuch as they are not precipitated by rennet or acid. Sweet whey contains about 4.9% of lactose, 0.8% of protein, 0.5% of minerals, 0.2% of milk fat, and 0.2% lactic acid.

The whey protein can be separated from whey or sweet whey by means of ultrafiltration (membrane technique, mean pore size: 25 to 100 kDalton). This “de-proteinized” whey consists to 95% of water and can be further processed to a powder by spray drying. This powder is here referred to as whey permeate. In the present invention, the use of whey permeate obtained from sweet whey is preferred. Its average components are 84.9% of lactose, 4.5% of protein, 0.1% of fat, and 7.5% of minerals. The remainder is water that has not been separated. These quantities are average values that may vary by 5-10% (relatively) depending on the method of preparation. Whey permeate can be used as such, e.g. in form of a powder, or, after (partial) hydrolysis of the lactose, as a syrup or a powder.

In a further embodiment according to the present invention, a microencapsulation of the pharmaceutical composition comprising calcium lactate and whey permeate has proved particularly advantageous. The microencapsulation may be achieved, e.g., as described in the patent application DE 198 54 749 A1 and DE 100 08 880 A1 and the utility model DE 296 23 285 U1. Thereby, the compound is, for example, firmly enclosed within a capsule made of a polysaccharide, such as, e.g., alginate. In order that the capsule material, which might possibly be indigestible, does not prevent the release of the compound and thereby its nutritional and physiological utilization, a digestible component, such as starch, may be added to the capsule. By adept choice and/or combination of the soluble and insoluble capsule components, the release of the microencapsulated pharmaceutical composition in various regions of the intestinal tract may thus be selectively controlled. A gradated release in the intestine, e.g. a release of 50 to 80% by weight, preferably of 60 to 70% by weight, in particular of 62.5 % by weight in the small intestine, and a release of 20 to 50% by weight, preferably of 30 to 40% by weight, in particular of 62.5% by weight in the large intestine is a possible manner of selective release. A further advantageous effect may be achieved by a longer storage life due to protection of the encapsulated compound, e.g., from environmental effects.

The pharmaceutical composition is preferably dosed in such a way that the dose, per administration amounts to 1.0 g to 15.0 g, preferably 5.0 g to 10 g, most preferably 6.0 to 9.0 g. Administration occurs advantageously in dosages of once to six times per day, a dosage of once to four times per day being preferred. Oral administration has proved advantageous, in particular in the quantities given above in 100 ml to 300 ml, preferably in 125 ml or 200 ml of a drink preparation. However, the actual dosage interval and dose depend upon factors such as, e.g., condition, age, weight, and/or sex of the treated individual, which can vary from individual to individual. Particularly in the case of pregnant and breast feeding women, taking an increased amount of the pharmaceutical composition is advantageous. An additional advantageous influence may, for example, result from taking the preparation shortly before or during mealtimes. For the administration of the preparation to mammals in general, dosage is also effected depending upon the species and weight.

According to the present invention, the pharmaceutical composition comprising calcium lactate and whey permeate may be used for the production of foodstuffs, such as, e.g., dietary foodstuffs and/or dietary supplements, upon which is thereby conferred the property of that they can effect a lowering of the triglyceride content when administered orally. Milk products or fruit juices enriched with the pharmaceutical composition are examples of such foodstuffs.

In the following, the invention is described in greater detail through experiments which are not intended to further limit the scope of the invention.

### EXPERIMENTS

Both pharmacological and nutrition-induced intervention trials on patients with an increased triglyceride level are above all time consuming, in order to detect changes of the metabolism, and require a high degree of discipline and motivation of the test persons or patients taking part in the studies. In part, these disadvantages can be lessened or eliminated by animal experiments, under the precondition that equivalent animal models are available. In contrast to most of the model animals described in the literature, which exhibit monogametic dominant hereditary disorders (Zucker rat, Kolesky rat, hHTG rat), the WOK.W rat, which has been studied, characterized and inbred at the University of Greifswald (Department of Laboratory Animal Science) since 1995, is an animal model that inherits the disorder polygenetically, and, amongst other things, develops an increased triglyceride level, with male animals expressing these symptoms more strongly. The specific properties of WOK.W rats are described, e.g., in:
P. Kovacs et al., Ann. N.Y. Acad. Sci., 1997, 827, 94-99;
P. Kovacs et al., Biochem. Biophsy. Res. Commun., 2000, 660-665;
J. van den Brandt et al., Int. J. Obesity, 2000, 24, 1618-1622;
J. van den Brandt et al., Metabolism 49, 2000, 1140-1144.

This animal model is suitable for studying an exogenous modulation as an intervention trial of this disease, especially since preliminary investigations prove that the incidence of the disease can be modulated, for example, by a diet that is rich in lipids (Table 1).

TABLE 1
Comparison of selected parameters of patients and WOK.W rats

| Parameter | WOK.W rat | Human |
|---|---|---|
| Obesity | +++¹ | +++ |
| Hypertriglyceridemia | +++ | +++ |
| Hypercholesterolemia | + | ++² |
| Dislipoproteinemia | ++ | +++ |
| Reduced HDL cholesterol | +³ | ++ |
| Hyperleptinemia | +++ | +++ |
| Glucose intolerance | ++ | +++ |
| Insulin resistance | ++ | +++ |
| Hyperinsulinemia | ++ | ++ |
| Hypertension | + | +++ |

¹+++ = very strongly pronounced,
²++ = strongly pronounced,
³+ = present

Trial Program

31 male WOK.W rats (from 8 litters) were divided, at an age of 4 weeks, into 4 groups, such that one sibling was represented in each group. Group 1 (n=8 animals) was fed and watered ad libitum and served as an untreated control group. Group 2 (n=7 animals) received drinking water to which had been added whey permeate (25 g sweet whey permeate/1000 ml drinking water), a daily water requirement of 20 ml on average being assumed as the basis of the calculation. Group 3 (n=8 animals) received drinking water to which had been added calcium L-lactate (5,6 g calcium L-lactate/1000 ml drinking water), and group 4 (n=8 animals) received whey permeate and calcium L-lactate in identical concentrations. The treatment trial began at an age of 4 weeks, i.e. before the development of the most important symptoms of the metabolic syndrome, and continued for 12 weeks, until an age of 16 weeks.

Parameters

Body weight, body length (for the calculation of the mody mass index, BMI), plasma glucose, and serum triglycerides were recorded every 2 weeks. At the end of the trial, serum was obtained and frozen, and furthermore the pancreas was prepared and frozen in liquid nitrogen or fixed in Bouin solution, to permit potential subsequent morphological examinations. Furthermore, the pararenal adipose tissue was prepared and weighed.

Plasma glucose was measured enzymatically using a glucose analyzer. Lipid metabolism parameters were determined enzymatically with an automated analyzer for clinical chemistry, with kits (Boehringer Mannheim: triglycerides with TG GPO-PAP), according to the supplied instruction manuals.

The pathological threshold values lie >8.4 mmol/l for plasma glucose, and >2.3 mmol/l for serum triglycerides.

The results are represented as mean values ±SEM and the statistical significance was calculated using Student's t-test.

Results

Body mass (as an expression of obesity) is not influenced by the chosen form of treatment, just as the BMI and the plasma glucose concentration do not change.

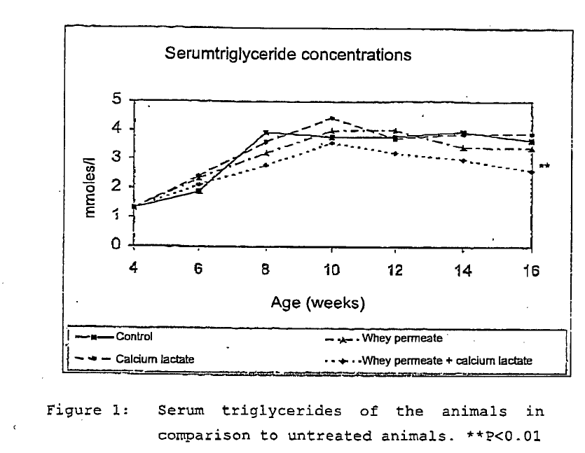

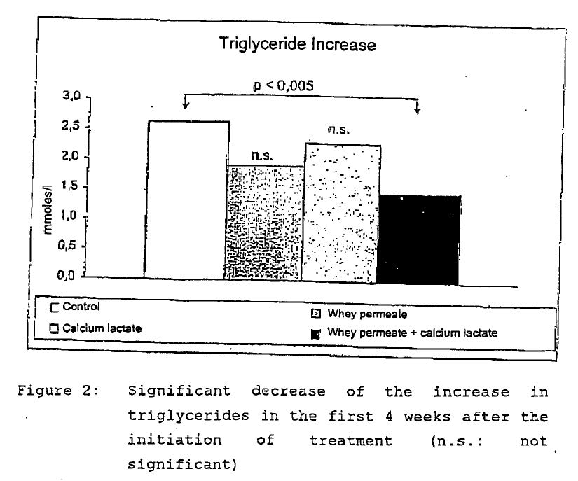

The pathogenetically significant serum triglycerides increase in all groups of test animals (FIG. 1) and exceed the pathological threshold value at an age of 6-8 weeks. The increase in TGs (FIG. 2) and the absolute TG concentrations are significantly lower in test group 4, although normal values could not be reached in any group. At no time did the singular treatment of the animals modulate the TG concentrations (FIG. 1).

The results of the parameters studied were summarized in Table 2, which was assembled at the end of the trial.

TABLE 2
Summary of the results of the feeding trial (male WOK.W rats, 16 weeks old)
after termination of dietary supplementation;

| | | | | | Whey permeate + |
|---|---|---|---|---|---|
| Parameter | Dimension | Control | Whey permeate | Calcium lactate | calcium lactate |
| Number | N | 8 | 7 | 8 | 8 |
| Body mass | G | 394.0 ± 10.2 | 406.3 ± 9.2 | 394.3 ± 10.6 | 385.0 ± 23.5 |
| BMI | g/cm² | 0.78 ± 0.01 | 0.76 ± 4.01 | 0.77 ± 0.01 | 0.75 ± 0.02 |
| Adipose | G | 11.50 ± 0.60 | 11.45 ± 0.57 | 10.01 ± 0.71 | 10.25 ± 1.06 |
| tissue | | | | | |
| Blood sugar | mmoles/l | 5.61 ± 0.11 | 6.05 ± 0.26 | 6.10 ± 0.21 | 5.64 ± 0.28 |
| Triglyceride | mmoles/l | 3.65 ± 0.18 | 3.39 ± 0.51 | 3.88 ± 0.22 | 2.60 ± 0.28^{a} |

^{a}P < 0.01,
^{b}P < 0.0001

Conclusions

The study proves that the combined treatment can significantly influence lipid metabolism parameters in rats with an increased triglyceride level. This opens the possibility of prophylaxis or treatment of hypertriglyceridemia and/or symptoms thereof, by combined administration of calcium lactate and whey permeate to mammals, in particular to humans.

1. Use of calcium lactate, in particular calcium L-lactate, and whey permeate, for the preparation of a pharmaceutical composition for the prophylaxis or treatment of an increased triglyceride level in a mammal, in particular in a human.

2. Use of calcium lactate, in particular calcium L-lactate, and whey permeate, for the preparation of a pharmaceutical composition for the prophylaxis or treatment of arteriosclerosis, coronary insufficiency, occlusive arterial diseases, myocardial infarction, xanthomata, abdominal complaints, spleen and liver enlargement, pancreatitis, retinal lipemia, and/or stroke, and/or diseases or disorders connected therewith in a mammal, in particular in a human.

3. The use according to claims 1 or 2, wherein the pharmaceutical composition serves as a simultaneous improvement of bone structure and/or as simultaneous prophylaxis and/or treatment of osteoporosis.

4. The use according any one of claims 1 to 3, wherein the whey permeate is obtained from sweet whey.

5. The use according any one of claims 1 to 4, wherein the whey permeate is hydrolyzed.

6. The use according any one of claims 1 to 5, wherein the whey permeate is partially hydrolyzed.

7. The use according any one of claims 1 to 6, wherein the composition is microencapsulated.

8. The use according any one of claims 1 to 7, wherein the composition is intended for oral administration.

9. The use according any one of claims 1 to 8, wherein the dose per administration is 1.0 g to 15.0 g, preferably 5.0 g to 10.0 g, most preferably 6.0 g to 8.0 g.

10. The use according any one of claims 1 to 9, wherein the calcium lactate is obtained by fermentation.

11. The use according any one of claims 1 to 10, wherein the composition furthermore contains additives that are suitable for the preparation of foodstuffs, in particular dietary foodstuffs and/or dietary supplements.

12. The use according any one of claims 1 to 11, wherein the composition furthermore comprises pharmaceutically acceptable additives and/or carrier materials.

13. The use according any one of claims 1 to 12, wherein the composition is in the form of a solid, as a lozenge, powder or granulate, or in the form of a liquid, as a sirup or juice.

14. Pharmaceutical composition comprising calcium lactate, in particular calcium L-lactate, and whey permeate.
